(19) United States
(12) Patent Application Publication
(10) Pub. No.: US 2014/0155359 A1
(43) Pub. Date: Jun. 5, 2014

(54) FORMULATION OF TRANSPARENT AND NUTRITIVE MICROEMULSIONS

(71) Applicants: Guy Broze, Grace Hollogne (BE); Claire Pucci, Croissy Sur Seine (FR); Adeline Navarro, Vourles (FR)

(72) Inventors: Guy Broze, Grace Hollogne (BE); Claire Pucci, Croissy Sur Seine (FR); Adeline Navarro, Vourles (FR)

(73) Assignee: PRAYON, Engis (BR)

(21) Appl. No.: 14/131,085

(22) PCT Filed: Jul. 13, 2012
(86) PCT No.: PCT/EP2012/063836

§ 371 Date: Feb. 21, 2014

(30) Foreign Application Priority Data
Jul. 15, 2011 (BE) 2011/0456

(51) Int. Cl. A23L 2/52; A23L 1/303; A23L 1/302; A61K 9/107

(52) U.S. Cl. CPC A23L 2/52; A61K 9/1075; A23L 1/303; A23L 1/302
USPC 514/167; 514/725; 514/458

(57) ABSTRACT

A clear and nutritive microemulsion comprising an aqueous phase in which at least one liposoluble active ingredient is dispersed, a first surfactant included in the group consisting of non-ionic surfactants with a high HLB and non-ionic surfactants with medium HLBs; and a second surfactant, characterized in that said second surfactant is chosen from the group consisting of anionic surfactants which have an HLB ≧25.

The present invention relates to the field of microemulsions.

Microemulsions are at the forefront of technology and have been under development for several years in many fields such as cosmetology and pharmacology. The food sector has, at present, been affected to only a small degree by this innovative process which, being as yet poorly controlled, tends to frighten the consumer. However, the process of microemulsion is gradually beginning to make an appearance on the food market in certain very specific cases such as, for example, the protection of flavourings, in order to prevent the volatilisation thereof or to control the spread thereof, or the formulation of transparent drinks.

A microemulsion is an isotropic dispersion of two immiscible phases, one aqueous and the other organic. Typically, a microemulsion comprises a minority dispersed phase and a majority continuous phase. When the dispersed phase is lipophilic, the microemulsion is said to be “oil-in-water” (0/W) and, conversely, if the continuous phase is lipophilic, it will be a “water-in-oil” (W/O) microemulsion.

The real advantage of microemulsions lies in their high thermodynamic stability, which is explained by the confinement of active ingredients of the dispersed phase inside very small aggregates called micelles. In order to create micelles, it is necessary to add a sufficient quantity of surface-active agents, which place themselves at the interface between the oily phase and the polar aqueous phase. When the concentration of surface-active agents increases and exceeds the critical micelle concentration (CMC), the surfactants self-organise themselves into labile domains called micelles, in which the dispersed phase takes refuge. This is then a microemulsion. It is the chemical nature of the surfactant molecules which will determine the direction of the 0/W or W/O microemulsion.

A microemulsion differs from a simple emulsion by its thermodynamic stability, which means that it should not degrade over time either in terms of transparency or in terms of micellar system. A microemulsion is transparent because the size of the drops of the dispersed phase is smaller than 100 nm. This transparency constitutes the strongest point of microemulsions for the foodstuffs field. In short, a microemulsion is thermodynamically stable and is transparent in nature.

An emulsion is generally not recommended in the foodstuffs field, in particular in the field of the production of nutritive drinks, given that an emulsion is not thermodynamically stable and will therefore not necessarily be transparent. Indeed, the diameter of the particles formed, at the time of emulsion, is greater than 100 nm. The incorporation of liposoluble active ingredients into an emulsion probably lieds to the obtaining of an emulsion in which said active ingredients are at risk of degrading over time. For that reason, it is not recommended to use an emulsion in the foodstuffs field.

A microemulsion can be administered to human beings and to livestock or competition animals through the formulation of microemulsions enriched with nutrients such as vitamins or antioxidants, which are easily and directly absorbed by the organism. The simplest form of marketing microemulsions enriched with liposoluble active ingredients (for example vitamins) lies in the formulation of transparent drinks with nutritional values.

More particularly, the invention relates to a clear and nutritive microemulsion which comprises an aqueous phase in which at least one liposoluble active ingredient is dispersed, a first surfactant included in the group consisting of non-ionic surfactants of high HLB and non-ionic surfactants of medium HLB; and a second surfactant.

A clear and nutritive microemulsion is known, for example, from document US 20070087104. Such a microemulsion is to be used in foods and in drinks by incorporating liposoluble active ingredients such as vitamins, antioxidants and/or flavourings into a microemulsion. It comprises a ternary system of surfactants comprising a surfactant of high HLB, a surfactant of medium HLB and a surfactant of low HLB. The surfactants used are chosen from non-ionic and anionic surfactants.

The formation of a conventional microemulsion requires strict operating conditions (such as, for example, homogenisation under high pressure), a not insignificant quantity of surfactants, or the addition of a co-solvent, such as ethanol or propylene glycol, which can lead to a final product that is free from taste. Moreover, the costs associated with these disadvantages are substantial.

The clear and nutritive microemulsion according to document US 20070087104 is obtained under less strict conditions without a co-solvent and with a lower dose of surfactants. However, this document teaches that it is necessary to use a surfactant of low HLB which enables the formation of an oily phase with the liposoluble active ingredient to reduce the total surfactant content of the microemulsion and to facilitate the steps of forming the microemulsion.

Moreover, the microemulsion according to document US 20070087104 is not stable when it is exposed to the heat of the surrounding medium. The microemulsion, for example contained in a drink, is easily and regularly exposed to an increase in temperature of the medium surrounding it, either during its transport and/or storage or when it is put on the market. During this cycle (transport and/or storage and/or putting on the market), it is important to be able to do without strict temperature conditions in order to preserve the stability of the active ingredients contained in the microemulsion. The stability, and therefore the shelf life, of the microemulsion is therefore essential to be able to exploit microemulsions commercially and industrially in a financially viable manner in the foodstuffs field, where profit margins are more limited than in the cosmetics industry, for example.

Generally, the road transport of drinks enriched with liposoluble active ingredients in the form of microemulsions involves the use of refrigerated lorries, the associated costs and the environmental impact of which cannot be disregarded on an industrial scale. Moreover, keeping (storing) such enriched drinks at an industrial site or in a warehouse also requires precautions in order to keep the product protected from an increase in temperature of the surrounding medium (due, for example, to prolonged exposure of the product to the sun). Controlling the temperature to which such enriched drinks are exposed is nowadays an essential element for preventing the product from becoming unstable and affecting public health. The instability of the microemulsion firstly can cause the food product to have an appearance that is not very appetising, and also promotes the degradation of the active ingredient, which is no longer protected and may therefore be degraded by the oxidising agents present in the water.

Within the meaning of the present invention, the term “HLB” is understood as being an empirical expression which expresses the hydrophilic and hydrophobic (or lipophilic) relationship of a surfactant. More precisely, the hydrophilic-hydrophobic balance (HLB) of a surfactant expresses the properties of the surfactant in question. A surfactant will therefore have a greater affinity for water if the HLB balance is high (hydrophilic nature) and, conversely, a surfactant will have a lower affinity for water (lipophilic or hydrophobic nature) when its HLB value is low.

The determination of the HLB value of a surfactant depends on the type of surfactant in question. As a function thereof, there are therefore two measuring methods, one for non-ionic surfactants and another for anionic surfactants.

The first calculation method allows an arbitrary scale for non-ionic polyethoxylated surfactants to be defined. The calculation method can be as follows:

HLB=20×Mh/M

The empirical formula allowing the HLB value of a non-ionic surfactant to be calculated comprises the ratio between the molecular mass of the hydrophilic group of the non-ionic surfactant in question (Mh) multiplied by 20 (molar mass of the ethoxylated group [(—CH₂—CH₂—O—)ₙ] of the non-ionic surfactant) and the molecular mass of the surfactant in question (M).

Starting from this empirical formula, an arbitrary scale is therefore defined and has HLB values comprised between 0 and 20. An HLB value of zero corresponds to a completely lipophilic surfactant, and an HLB value of 20 corresponds to a completely hydrophilic surfactant. Non-ionic surfactants are therefore classified according to this empirically established scale. A distinction is made, therefore, between non-ionic surfactants of low, medium and high HLB.

A surfactant of low HLB has an HLB value of from 0 to 6. A surfactant of medium HLB has an HLB value of from 6 to 14, and a surfactant of high HLB has an HLB value of from 14 to 20.

The second calculation method is based on the Davies method and takes into account the number of hydrophilic and lipophilic groups of the anionic surfactant in question. This calculation method allows an arbitrary scale for anionic surfactants to be defined.

HLB=7+Σ_{number of hydrophilic groups(+)}+Σ_{number of lipophilic groups(-)}

Table 1 shows different HLB values for various hydrophilic and lipophilic groups.

TABLE 1

| | HLB VALUES |
|---|---|
| HYDROPHILIC GROUPS | |
| —OSO₃Na | 38.7 |
| —CO₂K | 21.1 |
| —CO₂Na | 19.1 |
| SULFONATE | 11 |
| ESTER (SORBITAN RING) | 6.8 |
| ESTER (FREE) | 2.4 |
| —CO₂H | 2.1 |
| —OH (FREE) | 1.9 |
| —O— | 1.3 |
| —OH (SORBITAN RING) | 0.5 |
| —(CH₂—CH₂—O)— | 0.33 |
| LIPOPHILIC GROUPS | |
| —CH—, —CH₂—, —CH₃, ═CH— | 0.475 |

This method allows anionic surfactants to be classified relative to an arbitrary value, here 7, which is considered to be neutral. The hydrophilic groups have a positive contribution in the formula in question and the lipophilic groups have a negative contribution. Therefore, these two contributions influence the HLB value of an anionic surfactant. Let us consider, for example, SDS, which has more hydrophilic groups than lipophilic groups. This means that it will have a high HLB value (HLB_{SDS}=40) and that it will be more soluble in water than in oil.

Finally, the HLB value of a non-ionic or anionic surfactant allows the solubility of the surfactant in water or in oil to be indicated and therefore the direction of the emulsion (water-in-oil or oil-in-water) to be determined. For this reason, a non-ionic surfactant of high HLB will have a greater affinity for water and will therefore be more soluble in water than in oil and vice versa for surfactants of low HLB, which will be more soluble in oil. A surfactant that is more soluble in water than in oil will influence the direction of the “oil-in-water” emulsion and vice versa for a surfactant that is more soluble in oil than in water. This mechanism is similar for anionic surfactants, classified according to the “Davies” method.

Within the meaning of the present invention, the expression “clear and nutritive microemulsion” is understood as meaning a drink, an aqueous phase or a concentrated aqueous-based microemulsion to be added to drinks or to any other aqueous phase.

The expression “drinks enriched with liposoluble active ingredients” is therefore understood as meaning a drink containing a microemulsion, which is ready for consumption, having the above-mentioned stability conditions. This drink can be a pharmaceutical vitamin drink, a drink such as fizzy drinks, lemonade, water, fruit juice, or soups or sauces or any other partially liquid and aqueous foodstuff such as dairy products.

The terms “stable” or “stability” of a microemulsion within the meaning of the present invention are understood as meaning a microemulsion having chemical stability and physical or thermodynamic stability.

The “chemical stability” of a microemulsion within the meaning of the present invention is defined as the resistance of the active ingredient contained in the microemulsion to degradation, in particular to oxidation. The chemical stability can therefore be verified by analytical methods such as, for example, HPLC chromatography coupled with a UV detector, as explained below.

The “physical stability” or “thermodynamic stability” of a microemulsion within the meaning of the present invention is determined by its optical clarity. The optical clarity of a dispersed solution is assessed with the naked eye. When the size of the micelles is smaller than 100 nm (which corresponds to the wavelength of visible light), they are no longer visible to the naked eye. At that moment, the microemulsion is optically clear and therefore transparent to the naked eye. The optical clarity according to the present invention was assessed on a scale of 1 to 4. A solution classified as 4 on the scale in question defines an opaque solution, a solution classified as 3 has opalescent cloudiness, a solution classified as 2 has slight cloudiness, and a solution classified as 1 is transparent, that is to say that it is possible to look through it without noticing particles or residues. The physical or thermodynamic stability can also be measured by means of quasielastic light scattering (DLS), as explained below.

Generally, a thermodynamically stable microemulsion is therefore transparent, because the micelles contained in the microemulsion have a micelle size of less than 100 nm. The size of the micelles is governed by the so-called “natural curvature” of the micelle walls. This natural curvature depends on the ratio of the sizes (in reality, hydrodynamic volumes) of the hydrophilic and lipophilic portions of the system of surface-active agents and is influenced by the presence of the surfactants present in the system.

The object of the present invention is to remedy the disadvantages of the prior art by providing a stable microemulsion which withstands an increase in the temperature of the surrounding medium when it is transported and/or stored and/or put on the market. The microemulsion according to the present invention in fact exhibits chemical stability of the liposoluble active ingredients associated with the microemulsion and physical stability of the formulation, even when it is exposed to an increase in the temperature of the surrounding medium. The microemulsion developed therefore has a longer shelf life as compared to a known microemulsion and at the same time enables the costs associated with the preservation of the microemulsion when it is transported and/or stored or put on the market to be reduced.

In order to solve this problem, there is provided according to the invention a microemulsion as indicated above wherein said second surfactant is chosen from the group consisting of anionic surfactants having an HLB 25.

Within the meaning of the present invention, the expression “anionic surfactants having an HLB ≧25” is also understood as meaning anionic surfactants having preferably an HLB ≧26, more preferably an HLB ≧27, advantageously an HLB ≧28, preferably an HLB ≧29, more advantageously an HLB ≧30, preferably an HLB ≧31 and more preferably an HLB ≧32.

Within the scope of the present invention, and surprisingly, it has been shown that the addition of such an anionic surfactant having an HLB value ≧25 makes it possible to obtain a clear and (chemically and thermodynamically) stable microemulsion which withstands an increase in the temperature of the surrounding medium.

The presence of the anionic surface-active agent in fact increases the natural curvature of the micelle walls of the non-ionic surfactant of high HLB or of medium HLB and accordingly further promotes the reduction in the size of the micelles in the microemulsion. More precisely, said anionic surfactant leads to the formation of micelles of elongate form, which have a tendency to readily dissolve a large quantity of liposoluble active ingredients.

Surprisingly, the presence of an anionic surfactant allows the diameter of said micelles to be reduced up to 3 nm without affecting the (chemical and thermodynamic) stability of the microemulsion so formed and of the incorporated liposoluble active ingredients. The composition of the system of surface-active agents is therefore important, because it governs the size of the micelles in which the lipophilic active ingredients will be dissolved.

If, on the one hand, the size of the micelles is too large (diameter greater than 100 nm), they will scatter visible light and the product will not be transparent (not thermodynamically stable) and, on the other hand, if the micelles are too small (diameter less than approximately 3 nm), their ability to incorporate lipophilic substances will be limited, which likewise leads to a cloudy or unstable system.

It has been found that the non-ionic surfactant of high HLB or of medium HLB interacts synergistically with said anionic surfactant. The anionic surfactant has a negative charge, which has a large hydrodynamic volume when it is hydrated, and a hydrophobic group of smaller volume. The surfactant therefore has the shape of a pear, the body of which is constituted by the hydrated anionic group and the tail by the hydrophobic chain. The addition of said anionic surfactant to a solution in the presence of a non-ionic surfactant of lower HLB will have the effect of reducing the natural curvature of the non-ionic surfactant in question and thus forming a microemulsion that is even more stable as compared to a known microemulsion. Moreover, in view of the low impact of the temperature on the hydration of the anionic group, the presence of the anionic surfactant accordingly allows better stability of the microemulsion so formed to be ensured in the event of an increase in the temperature of the surrounding medium.

It should be added that the presence of an anionic surfactant does not contribute to the chemical stability of the incorporated lipophilic active ingredient. For this reason, it is particularly surprising and remarkable to have been able to retain the (chemical and physical) stability of the microemulsions so formed and of the incorporated active ingredients.

Moreover, it has been found, wholly surprisingly, that it is possible to obtain a particularly stable and transparent microemulsion using two surfactants of high HLB or using one surfactant of medium HLB and one surfactant of high HLB without having to use a carrier oil, acetone or a surfactant of low HLB (value from 0 to 6), contrary to the teaching of the prior art, in particular of document US 20070087104.

The presence of a non-ionic surfactant of high HLB which can contain polyethylene oxide (PEO) allows an osmotic barrier to be formed in the microemulsion. The presence of a non-ionic surfactant of high HLB in the microemulsion according to the invention therefore allows the diffusion of oxygen into the micelles to be slowed down and the oxidation of the active ingredients contained in the microemulsion thus to be reduced. Consequently, the chemical stability of the active ingredients is retained over time.

The presence of a non-ionic surfactant of medium HLB in the microemulsion also allows the natural curvature of the micelle walls to be adjusted so as to increase their ability to dissolve the active ingredient. This effect is enhanced in the presence of the anionic surfactant, which has a negative charge and a hydrophobic group. The presence of an anionic surfactant in fact considerably enhances the increase in the natural curvature of the micelle walls and accordingly enables the size of the micelles to be reduced up to 3 nm.

Advantageously, the microemulsion according to the invention further comprises a third surfactant chosen from the group consisting of non-ionic surfactants of high HLB when the first surfactant is a non-ionic surfactant of medium HLB, or from the group consisting of non-ionic surfactants of medium HLB when the first surfactant is a non-ionic surfactant of high HLB.

Consequently, the microemulsion may therefore contain three surfactants: a non-ionic surfactant of high HLB, a non-ionic surfactant of medium HLB, and an anionic surfactant of HLB ≧25.

The binary system comprising two surfactants or the ternary system comprising three surfactants therefore comprises a judicious mixture of surface-active agents of high and/or medium HLB in the presence of an anionic surfactant which has an HLB value ≧25. The mixture of said surface-active agents therefore forms a system characterised by a synergy between the various surfactants, leading to the formation of micelles having sizes which can reach 3 nm, while at the same time ensuring a good dissolving power for the incorporated lipophilic active ingredients.

In a particular embodiment, the microemulsion according to the invention is characterised in that the first and/or the third non-ionic surfactant of high HLB is chosen from the group consisting of polyoxyethylene sorbitan esters, in particular sorbitan monododecanoate poly(oxy-1,2-ethanediyl) (Tween 20 or Polysorbate 20), and alkyl polyglucosides (APGs). Tween 20 or Polysorbate 20 is a low-viscosity yellow liquid which is of food grade, in particular at European level, and has the following structure of formula (I):

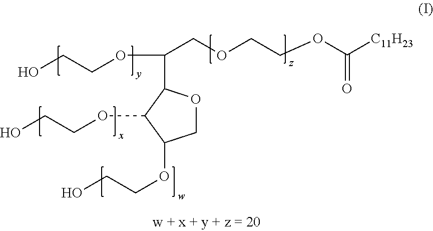

Tween 20 or Polysorbate 20 has an HLB of 16.7. Accordingly, when the microemulsion comprises said non-ionic surfactant of high HLB and an anionic surfactant, Tween 20 is considered to be the principal surfactant (from 70 to 95% by weight) and the anionic surfactant is considered to be a co-surfactant (from 5 to 30% by weight). Tween 20 is described as the principal surfactant because it is present in a larger quantity relative to the anionic surface-active agent and is therefore responsible for the direction of the oil-in-water emulsion.

In a binary system comprising a non-ionic surface-active agent of medium HLB and an anionic surfactant, the non-ionic surfactant of medium HLB is then the principal surfactant and the anionic surfactant is the co-surfactant in the microemulsion in question.

In a ternary system comprising non-ionic surfactants of high and medium HLB and an anionic surfactant of HLB ≧25, the principal surfactant is the non-ionic surfactant of high HLB because it is present in a larger quantity (responsible for the direction of the oil-in-water emulsion), and the co-surfactants are the non-ionic surfactant of medium HLB and the anionic surfactant, which act principally on the adjustment of the natural curvature of the micelle walls.

Moreover, since the majority of active ingredients are sensitive to oxidation, it is advantageous for the wall of the micelles to slow down the diffusion of oxygen into the micelles. The presence of non-ionic surfactants of high HLB containing polyethylene oxide (PEO) is therefore desirable, allowing the risk of oxidation of the liposoluble active ingredients to be reduced still further.

Preferably, the microemulsion according to the invention is characterised in that the first and/or the third non-ionic surfactant of medium HLB is chosen from the group consisting of sorbitan esters, in particular sorbitan laurate, polyoxyethylene (20) sorbitan monolaurate, polyoxyethylene (20) sorbitan monopalmitate, polyoxyethylene (20) sorbitan monostearate, polyoxyethylene (20) sorbitan monooleate, polyoxyethylene glycol sorbitan laurate, hexaethylene glycol sorbitan monooleate, polyoxyethylene sorbitan stearate, decaglyceryl monooleate, decaglyceryl dioleate, polyoxyethylene sorbitan tristearate, monodehydrosorbitol monooleate, sorbitan monolaurate, sorbitan monopalmitate and sorbitan laurate (Span 20).

Span 20 has an HLB of 8.6 and is sold in the form of a particularly viscous yellow-orange liquid. The structure of Span 20 is shown by formula (II):

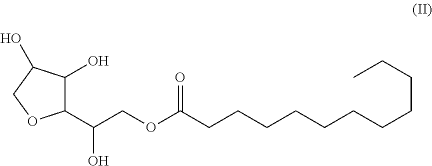 [SMILES: CCCCCCCCCCCC(=O)OCC(O)C1OCC(O)C1O]

When the microemulsion comprises only a surfactant of medium HLB and an anionic surfactant of HLB ≧25, the surfactant of medium HLB is the principal surfactant, which is responsible for the direction of the emulsion, and the anionic surfactant is the co-surfactant. It should be added that the anionic surfactant of HLB ≧25 is present in a smaller quantity relative to the non-ionic surfactant of medium HLB and cannot be present in a large quantity for reasons relating to the taste and chemical stability of the lipophilic active ingredient.

According to a preferred embodiment of the invention, the second anionic surfactant of HLB ≧25 is chosen from the group consisting of sodium dodecyl sulfate (SDS or SLS), alcohol sulfates, alcohol ethoxysulfates, alkylsulfonates and carboxylic acids and their salts, in particular gluconic acid and its derivatives.

In a particularly advantageous embodiment, the microemulsion according to the present invention is characterised in that the liposoluble active ingredient is a vitamin chosen from the group consisting of vitamin D, vitamin K, vitamin A and vitamin E.

The advantage of being able to incorporate at least one vitamin into the microemulsion enables the formulation of nutritive drinks. The presence of vitamins in, for example, a nutritive drink allows vitamins to be taken in a simple manner. This intake is important for the human or animal organism, since it allows potential vitamin deficiencies in the organism to be prevented. For several years, nutritionists have been observing vitamin D deficiencies in human beings and are beginning to be concerned thereby, particularly vitamin D deficiencies. Vitamin D exists in various forms, the best known of which are vitamin D2 or ergocalciferol, which is of plant origin and is found in the majority of foods, and vitamin D3 or cholecalciferol, which is of animal origin and is synthesised by the skin on exposure to the UV rays of the sun.

Vitamin D is a liposoluble vitamin which is synthesised by the organism itself. However, in countries that are not very sunny, and at certain times of the year, a vitamin D deficiency can occur. In children or adolescents this can manifest itself as rickets, while in adults it leads to osteoporosis. The primary role of vitamin D is in fact to promote the fixing of calcium to the bones, which allows them to grow and solidify. Moreover, it is known to facilitate the intestinal absorption of calcium and phosphorus. It is within this context that it appears highly valuable to incorporate vitamin D into a microemulsion, for example.

Moreover, in a particular embodiment, the microemulsion according to the invention further comprises an antioxidant, preferably chosen from the group consisting of caffeic acid, pomegranate plant extracts, rosemary plant extracts, rutin, vitamin E, polyphenols, preferably quercetin, and mixtures thereof.

The presence of an antioxidant according to the invention allows the risk of oxidation of the liposoluble active ingredient to be reduced still further and thus confers enhanced chemical stability upon the microemulsion.

Advantageously, the microemulsion according to the invention can likewise further comprise an oily phase, despite the fact that it is not necessary, allowing the process of using the microemulsion to be facilitated. When the lipophilic active ingredient is to be added in a small quantity relative to the final volume of the microemulsion, prior dilution thereof in an oily carrier phase, preferably glycerol oleate, allows the precision of the quantity added to be improved and the reproducibility of the microemulsion to be improved, in particular when it is prepared in an industrial quantity by industrial devices of which the accuracy on a very small scale sometimes leaves something to be desired.

Other embodiments of the microemulsion according to the invention are indicated in the accompanying claims.

The present invention also relates to a process for the production of a microemulsion obtained according to the invention. The process for the production of the microemulsion comprises the following steps:

a) mixing, with stirring, at least two surfactants with at least one liposoluble active ingredient until a homogeneous solution is obtained, and
b) mixing, with stirring, said homogeneous solution with an aqueous phase until a microemulsion is obtained.

The production process according to the invention therefore permits the use of devices that are not complex and implementation of the process that is particularly simplified. Moreover, handling associated with the maintenance of production devices at ambient temperature considerably reduces these constraints from both an ecological and an economic point of view during the implementation of the process for the production of said microemulsion.

As can be noted in the process, according to the present invention all the surfactants are added simultaneously to the liposoluble active ingredient to form a homogeneous solution, which is then mixed with the aqueous phase. The process is therefore very simple and does not require restrictive temperature control or complex devices.

According to a preferred embodiment of the invention, the production process further comprises dilution of said microemulsion with an aqueous medium such as water, for example distilled water and/or food-grade water, optionally in admixture with other additives, for example ortho-phosphoric acid and/or citric acid, to form a nutritive drink enriched with liposoluble active ingredients.

Advantageously, the process according to the present invention further comprises the addition of an antioxidant, preferably chosen from the group consisting of caffeic acid, pomegranate plant extracts, rosemary plant extracts, rutin, vitamin E, polyphenols, preferably quercetin, and mixtures thereof.

Confinement of the lipophilic active ingredient in the micelles allows molecules having an antioxidant nature to be added to the micelles or to the periphery thereof. Caffeic acid, pomegranate plant extracts, rosemary plant extracts, rutin, vitamin E, polyphenols, preferably quercetin, and mixtures thereof thus allow the chemical stability of the active ingredient, in particular vitamin D3, incorporated into the microemulsion to be improved spectacularly. Although the invention is not linked in any way to the exactitude of this mechanism, the excellent stabilisation of vitamin D by the antioxidants is without doubt linked to the fact that the vitamin D and the antioxidants are confined in or at the surface of the micelles. Accordingly, the relative concentration of antioxidant is high in the region of the core of the micelle and not in the aqueous phase.

Other embodiments of the process for the production of the microemulsion according to the invention are indicated in the accompanying claims.

The present invention also relates to the use of an anionic surfactant having an HLB ≧25 for the production of a clear and nutritive microemulsion.

Advantageously, according to the invention, said anionic surfactant is chosen from the group consisting of sodium dodecyl sulfate (SDS or SLS), alcohol sulfates, alcohol ethoxysulfates, alkylsulfonates and carboxylic acids and their salts, in particular gluconic acid and its derivatives.

Other forms of use are indicated in the accompanying claims.

The present invention also relates to a clear and nutritive drink comprising a microemulsion obtained according to the invention.

Advantageously, the drink according to the invention comprises an antioxidant which is preferably chosen from the group comprising quercetin, caffeic acid, pomegranate plant extracts, rosemary plant extracts, and mixtures thereof.

Other forms of the clear and nutritive drink comprising a microemulsion are indicated in the accompanying claims.

Other features, details and advantages of the invention will become apparent from the following description, which is given by way of non-limiting example and with reference to the examples.

A binary microemulsion is prepared according to the present invention by first simultaneously mixing:

a) from 70 to 0.98% by weight of Tween 20,
b) from 0.02 to 0.30% by weight of SDS,
c) from 0.002 to 0.02% by weight of vitamin D, and
d) from 0.002 to 0.02% by weight of quercetin
until a homogeneous solution is obtained.

The homogeneous solution is then mixed, with stirring, preferably for one hour, in order to obtain the microemulsion according to the binary system.

According to a preferred embodiment of the invention, the microemulsion can also be obtained by means of a ternary system of surfactants. In this preferred embodiment of the invention, the preparation of said microemulsion comprises the simultaneous mixing of:

a) from 70 to 0.95% by weight of Tween 20,
b) from 0.05 to 0.30% by weight of Span 20,
c) from 0.01 to 0.20% by weight of SDS,
d) from 0.002 to 0.02% by weight of vitamin D, and
e) from 0.002 to 0.02% by weight of quercetin
until a homogeneous solution is obtained, which solution is then stirred, preferably for one hour, in order to prepare the microemulsion comprising a ternary system of surfactants.

The method of preparing the microemulsion according to the invention makes it possible to avoid the use of an expensive and restrictive technique which requires steps of heating or homogenisation under high pressure. The production of the microemulsion developed is simple and economically viable since it is sufficient to simultaneously mix the surfactants with at least one active ingredient without having to use expensive air-conditioning conditions.

The use of an anionic surfactant having an HLB 25 in an at least binary system is therefore indispensable for the preparation of a clear and nutritive microemulsion which is capable of withstanding an increase in temperature of the surrounding medium and the shelf life of which is consequently increased in relation to a known microemulsion. Advantageously, the anionic surfactant which has an HLB 25 is preferably gluconic acid and its derivatives, such as sodium, potassium, calcium and ferrous gluconate or glucono delta-lactone.

Other formulations of microemulsions according to the invention are detailed in the examples and comparative examples described below.

Within the scope of the invention, the microemulsion developed can be used in the foodstuffs field, for example to produce a clear and nutritive drink. Said microemulsion is in fact transparent (optical clarity) and the size of the micelles is less than 100 nm, which is suitable for the formulation of nutritive drinks into which vitamins have been incorporated beforehand. This type of nutritive drink is further recommended for athletes or for individuals who perform a regular sporting activity. Physical activity leads to the loss of essential elements (vitamins, minerals, etc.) from the human organism. For this reason, such a microemulsion allows, for example, persons performing a sporting activity to replenish vitamins sweated out during intense physical exertion.

When the microemulsion is formed, it is necessary to be able to monitor several factors such as the presence and the rate of degradation of the active ingredient(s) incorporated into the microemulsion and the transparency of the solutions (optical clarity), which is evaluated via the size of the micelles.

Monitoring within the reaction medium of the presence or absence of the active ingredients previously incorporated into the microemulsion can be carried out by means of an analytical measuring technique called “high performance liquid chromatography” (HPLC). This technique is based on a difference in the affinity of each constituent of a mixture between the stationary phase of the column and its mobile phase eluting it. This difference in affinity in fact arises from the greater or lesser value of the polarity of each molecule of the formulation. Each of the components therefore has a retention time in the column which is characteristic thereof and permits its qualitative recognition according to the chromatograms of a library. Moreover, by preparing the calibration line of a given molecule at different known concentrations, which respect the Beer-Lambert law, it is possible to assay a constituent of a mixture in a quantitative manner, even if it is introduced at a concentration of the order of the ppm or part per million.

The HPLC apparatus used for these analyses is an Agilent 1100 Series having a Zorbax C18 column and a DAD detector (diode array UV detector). For each analysis, the temperature of the column remains the same, namely 30° C., the flow rate is kept constant at 1 ml/min and the injection volume is always 50 μl. The parameters to be fixed by the operator are the nature of the eluent (in the present case the methanol/water ratio), the analysis time (according to the retention times of the constituents to be detected) and therefore the method to be chosen.

The rate of degradation (ageing) linked to the chemical stability of the active ingredients previously incorporated into the microemulsion is also evaluated by HPLC. This ageing test enables to determine the most effective antioxidants for protecting the active ingredient(s) incorporated into the microemulsion and accordingly allows their shelf life to be further increased.

Each ageing test is carried out in a plastics bottle (of the 1.5 litre Charmoise water bottle type). The samples are first formulated to 100 g of solution and then diluted by 5 or 6 to obtain a total volume of approximately 500 or 600 ml in the bottles. There is thus a permanent flow of oxygen air above the solution, enabling the efficacy of the antioxidants to be tested. All of the bottles so created are stored in daylight and at ambient temperature.

Measurement of the size of the micelles generated during the formation of the microemulsion is carried out by means of a quasielastic (dynamic) light scattering (DLS) apparatus in order to verify whether said microemulsion comprises micelles and/or drops of oil which have a size smaller than 100 nm. This technique thus makes it possible to have a more precise idea of the exact terminology to be employed to describe the solutions created (microemulsions, emulsions or submicron dispersions). The measurement consists in transmitting a light beam onto the sample, which is contained in a glass cell. The light beam is scattered with a different intensity according to the size and concentration of the objects contained in the solution. A particle of small size scatters the light very little, whereas a large object scatters the light considerably. Accordingly, the detection range of the apparatus extends approximately from a nanometre to 1 μm. It is essential that the samples are diluted in order to avoid skewing the results. The micelles can interact with one another in two ways in solution, by repelling one another or by attracting one another. In both cases, a certain structuring of the objects results, which induces a modification of the scatter profile. The more concentrated the solution, the more these phenomena of micellar interactions are present. Accordingly, in order to obtain size distributions that are as correct as possible, the samples must be diluted.

In reality, the apparatus measures the light scattering coefficient as a function of the time and not directly the apparent diameter of the objects. During the analysis, the particles, animated with a Brownian motion, move in solution. For this reason, the measurements are carried out as a function of the time, and dynamic light scattering is measured. In order to relate the size of the particles to the diffusion coefficient, the software uses the Stokes-Einstein law, which assumes that the particles are spherical and that the movements of the molecules are translations.

Stokes-Einstein law:D=(k*T)/(6*π*η*R)

D: diffusion coefficient (measured by the apparatus)
k: Boltzmann constant
T: temperature of the medium
η: dynamic viscosity of the continuous phase (here water)
R: radius of the particle (or of the micelles)

The apparatus used within the scope of these studies is a Zetasizer nano series from Malvern instrument, which carries out the analyses at a fixed scattering angle of 173°. The measurements are processed by DTS nano software, which supplies the results in two distinct forms:
The cumulant method, which gives the Z-average of the apparent diameter and the polydispersity index (PDI). This is the most correct result and is therefore the one to be considered in the case of a size distribution having a single population. The PDI indicates whether the population in question is broad, that is to say very dispersed or fine. The smaller the PDI value, the narrower the population.
Distribution analysis, which is provided by means of an algorithm, which has a tendency to diverge. In order to avoid this divergence, the software carries out a correction, which skews the results slightly. However, this distribution analysis is found to be very advantageous because it allows not only the number of populations present within the sample to be determined, but also the scattered intensity value as a function of the apparent diameter of the objects.

Before each analysis, it must be ensured that the samples are filtered by means, preferably, of a filter of the nylon 0.22 μm type in order to avoid the presence of dust, which can induce a measurement artefact, namely the creation of a new population of large objects.

Example 1 illustrates a binary system of surfactants comprising Tween 20 or Polysorbate 20 (non-ionic surfactant of high HLB), SDS (anionic surfactant, HLB ≧25) and vitamin D3 as the liposoluble active ingredient.

### EXAMPLE 1

| Products | Quantity (g) | % by weight |
|---|---|---|
| Water | 597.53 | 99.57 |
| Tween 20 | 2.00 | 0.33 |
| SDS | 0.51 | 0.08 |
| Vitamin D3 | 0.07 | 0.01 |
| Total | 600.11 | 100.00 |

| | |
|---|---|
| DLS | ≈10 nm |

Tween 20 or Polysorbate 20 acts as the principal surfactant because it is the surfactant that is introduced in the greatest quantity (2 g). SDS is then designated the co-surfactant (0.51 g) and allows the curvature of the micelles to be adjusted. It should be added that SDS is an anionic surfactant having an HLB with a value of 40.

The presence of SDS in the microemulsion enables a micelle size of approximately 3 nm obtained by DLS to be achieved. This clearly shows that the solution obtained is a microemulsion and is therefore thermodynamically stable.

The present microemulsion was produced by mixing Tween 20 or Polysorbate 20, SDS and vitamin D3 for one hour, with stirring, until a homogeneous solution is obtained. Water is then added to the homogeneous solution, with stirring by means of a magnetic stirring rod, until a microemulsion is obtained. Finally, the microemulsion is diluted by 6 in distilled water.

Example 2 illustrates a binary system of surfactants comprising Span 20 (non-ionic surfactant of medium HLB), SDS (anionic surfactant) and vitamin D3 (active ingredient).

The present microemulsion was produced by first mixing distilled water with SDS to form a first mixture. A second mixture was then produced by mixing Span 20 and vitamin D3. The two mixtures are stirred (by means of stirrers) until the SDS and the vitamin D3 have dissolved completely to give a homogeneous solution. When the products contained in said mixture have dissolved completely, the first mixture containing distilled water and SDS is added gradually, still with stirring, to the second mixture. In the presence of Span 20 and SDS in the homogeneous solution, it is sometimes necessary to heat the solution, for example at a temperature of approximately 100° C., in order to activate the kinetics of the reaction.

### EXAMPLE 2

| Products | Quantity (g) | % by weight |
|---|---|---|
| Water | 97.49 | 82.96 |
| Span 20 | 0.99 | 0.84 |
| SDS | 18.99 | 16.16 |
| Vitamin D3 | 0.05 | 0.04 |
| Total | 117.52 | 100 |

Example 3 illustrates a ternary system comprising Tween 20, Span 20, SDS and vitamin D3.

The present microemulsion was produced in the same manner as that in example 2.

The size of the micelles, obtained by DLS, increases to 14.03 nm, which corresponds to a thermodynamically stable microemulsion.

### EXAMPLE 3

| Products | Weight (g) | % by weight |
|---|---|---|
| Tween 20 | 1.95 | 0.33 |
| Span 20 | 0.41 | 0.07 |
| Vitamin D3 | 0.05 | 0.01 |
| SDS | 0.18 | 0.03 |
| Demineralised water | 595.31 | 99.57 |
| Total | 597.90 | 100.00 |

DLS
14.03 nm

Example 4 illustrates a ternary system comprising Tween 20, Span 20, SDS, vitamin D and quercetin, which serves as an antioxidant in the microemulsion.

The method of producing the present microemulsion is identical to that described in example 2.

### EXAMPLE 4

| Products | Quantity (g) | % by weight |
|---|---|---|
| Water | 595.00 | 99.15 |
| Tween 20 | 3.72 | 0.62 |
| Span 20 | 0.65 | 0.11 |
| SDS | 0.65 | 0.11 |
| Quercetin | 0.03 | 0.005 |
| Vitamin D3 | 0.05 | 0.008 |
| Total | 600.1 | 100.00 |

| Transparency | Before dilution | After dilution | After heating |
|---|---|---|---|
| Example 4 | 2 | 1 | 1 |

In the presence of quercetin, the microemulsion is more chemically stable over time because its presence allows the diffusion of oxygen into the micelles to be slowed down still further.

The transparency of the microemulsion developed was evaluated before dilution of the microemulsion, after dilution of the microemulsion and after heating of the microemulsion. When the microemulsion is exposed to an increase in the temperature of the surrounding medium, it is found that the microemulsion is still transparent (1) and therefore retains its chemical and thermodynamic stability.

Example 5 illustrates a ternary system comprising Tween 20, Span 20, SDS and vitamin A (active ingredient).

### EXAMPLE 5

| Products | Quantity (g) | % by weight |
|---|---|---|
| Water | 389.80 | 97.37 |
| Tween 20 | 7.85 | 1.96 |
| Span 20 | 1.64 | 0.41 |
| SDS | 0.72 | 0.18 |
| Vitamin A | 0.31 | 0.08 |
| Total | 400.32 | 100.00 |

The present microemulsion was produced by preparing separately a first mixture containing water and SDS and a second mixture containing Span 20, Tween 20 and vitamin A. The two mixtures are then stirred until the SDS, on the one hand, and the vitamin A, on the other hand, have dissolved completely. When said products have dissolved completely, the first mixture is added to the second, still with stirring (by means of stirrers), until the microemulsion is obtained.

Example 6 illustrates a composition of a microemulsion comprising Tween 20, Span 20, SDS, vitamin A and rutin (antioxidant).

### EXAMPLE 6

| Products | Quantity (g) | % by weight |
|---|---|---|
| Water | 391.90 | 97.33 |
| Tween 20 | 7.82 | 1.94 |
| Span 20 | 1.63 | 0.40 |
| SDS | 0.70 | 0.17 |
| Vitamin A | 0.20 | 0.05 |
| Rutin | 0.41 | 0.10 |
| Total | 402.65 | 100.00 |

The method of producing the present microemulsion is identical to that described in example 5, except that the second mixture further comprises rutin.

Example 7 also illustrates a ternary system such as that in example 6, except that rutin has been replaced by quercetin. Moreover, the method of producing the present microemulsion is identical to that described in example 6.

### EXAMPLE 7

| Products | Quantity (g) | % by weight |
|---|---|---|
| Water | 389.20 | 97.35 |
| Tween 20 | 7.84 | 1.96 |
| Span 20 | 1.63 | 0.41 |
| SDS | 0.70 | 0.18 |
| Vitamin A | 0.20 | 0.05 |
| Quercetin | 0.20 | 0.05 |
| Total | 399.78 | 100.00 |

Example 8 illustrates a composition of a microemulsion comprising a ternary system (Tween 20, Span 20 and SDS) in the presence of vitamin E as the liposoluble active ingredient.

The present microemulsion was produced by preparing a first mixture containing water and SDS and a second mixture containing Span 20, Tween 20 and vitamin E. Said mixtures are then stirred (by means of stirrers) until the SDS and the vitamin E have dissolved completely. When said products have dissolved completely, the first mixture is added to the second, still with stirring. Finally, the mixture obtained is heated at 100° C. in order to obtain clarification of the microemulsion so that it is thermodynamically stable.

### EXAMPLE 8

| Products | Quantity (g) | % by weight |
|---|---|---|
| Water | 595 | 99.15 |
| Tween 20 | 3.76 | 0.63 |
| Span 20 | 0.74 | 0.12 |
| SDS | 0.49 | 0.08 |
| Vitamin E | 0.11 | 0.02 |
| Total | 600.10 | 100.00 |

Example 9 illustrates a composition of a microemulsion comprising Tween 20, Span 20, gluconic acid and vitamin D. In the present example, gluconic acid, partially “deprotonated” under the conditions of the example, is an anionic surfactant which therefore plays the same role as SDS in the microemulsion. It should be noted that gluconic acid has an HLB value of 33.2.

The present microemulsion was produced by preparing a first mixture containing water and gluconic acid and a second mixture containing Span 20, Tween 20 and vitamin D. Said mixtures are then stirred separately (by means of stirrers) until the gluconic acid and the vitamin D have dissolved completely. When said products have dissolved completely, the first mixture is added to the second, still with stirring. Finally, the mixture can be heated at 100° C. to obtain an even clearer microemulsion.

### EXAMPLE 9

| Products | Quantity (g) | % by weight |
|---|---|---|
| Water | 595.004 | 99.16 |
| Tween 20 | 3.848 | 0.64 |
| Span 20 | 0.350 | 0.058 |
| Gluconic acid | 0.811 | 0.135 |
| Vitamin D3 | 0.051 | 0.008 |
| Total | 600.064 | 100.00 |

Comparative example 1 illustrates the composition of a known microemulsion comprising a binary system of surfactants comprising Tween 20 as the principal surfactant and Span 20 as the co-surfactant.

The method of producing the microemulsion was carried out in the same manner as that described for the composition of the microemulsion illustrated in example 1.

The size of the micelles formed during the production of the microemulsion was measured by DLS and is between 10 and 50 nm.

It should be added that the presence of an anionic surfactant in the microemulsion according to the present invention, in particular of SDS, permits the formation of a microemulsion which withstands an increase in the temperature of the surrounding medium, contrary to the known microemulsions such as the binary system comprising Tween 20 and Span 20.

### COMPARATIVE EXAMPLE 1

| Products | Quantity (g) | % by weight |
|---|---|---|
| Water | 589.96 | 98.32 |
| Tween 20 | 8.51 | 1.42 |
| Span 20 | 1.50 | 0.25 |
| Vitamin D3 | 0.05 | 0.01 |
| Total | 600.03 | 100.00 |

| | |
|---|---|
| DLS | 10-50 nm |

Comparative example 2 illustrates the composition of a known microemulsion comprising Tween 20, Span 20, quercetin and vitamin D.

The transparency of the known microemulsion rises to 3 before dilution and between 1-2 after dilution. Therefore, when dilution is carried out, the microemulsion obtained is transparent. However, the known microemulsion does not withstand an increase in the temperature of the surrounding medium because the transparency after heating was evaluated at 4, which corresponds to an opaque solution.

### COMPARATIVE EXAMPLE 2

| Products | Quantities (g) | % by weight |
|---|---|---|
| Water | 594.97 | 99.15 |
| Tween 20 | 4.25 | 0.71 |
| Span 20 | 0.75 | 0.12 |
| Quercetin | 0.03 | 0.005 |
| Vitamin D3 | 0.05 | 0.008 |
| Total | 600.05 | 100.00 |

| Transparency | Before dilution | After dilution | After heating |
|---|---|---|---|
| Comparative | 3 | 1-2 | 4 |
| example 2 | | | |

Clearly the present invention is in no way limited to the embodiments described above, and modifications can be made thereto without departing from the scope of the accompanying claims.

1. Clear and nutritive microemulsion comprising an aqueous phase in which at least one liposoluble active ingredient is dispersed, a first surfactant included in the group consisting of non-ionic surfactants of high HLB and non-ionic surfactants of medium HLB; and a second surfactant, characterised in that said second surfactant is chosen from the group consisting of anionic surfactants having an HLB ≧25.

2. Microemulsion according to claim 1, further comprising a third surfactant from the group consisting of non-ionic surfactants of high HLB when the first surfactant is a non-ionic surfactant of medium HLB, or from the group consisting of non-ionic surfactants of medium HLB when the first surfactant is a non-ionic surfactant of high HLB.

3. Microemulsion according to claim 1, wherein said first and/or third non-ionic surfactant of high HLB is chosen from the group consisting of polyoxyethylene sorbitan esters, in particular sorbitan monododecanoate poly(oxy-1,2-ethanediyl) and alkyl polyglucosides (APGs).

4. Microemulsion according to claim 1, wherein said first and/or third non-ionic surfactant of medium HLB is chosen from the group consisting of sorbitan esters, in particular sorbitan laurate, polyoxyethylene (20) sorbitan monolaurate, polyoxyethylene (20) sorbitan monopalmitate, polyoxyethylene (20) sorbitan monostearate, polyoxyethylene (20) sorbitan monooleate, polyoxyethylene glycol sorbitan laurate, hexaethylene glycol sorbitan monooleate, polyoxyethylene sorbitan stearate, decaglyceryl monooleate, decaglyceryl dioleate, polyoxyethylene sorbitan tristearate, monodehydrosorbitol monooleate, sorbitan monolaurate, sorbitan monopalmitate.

5. Microemulsion according to claim 1, wherein said second anionic surfactant is chosen from the group consisting of sodium dodecyl sulfate (SDS), alcohol sulfates, alcohol ethoxysulfates, alkylsulfonates and carboxylic acids and their salts, in particular gluconic acid and its derivatives.

6. Microemulsion according to claim 1, wherein said anionic surfactant is chosen from the group consisting of gluconic acid and its derivatives such as sodium, potassium, calcium and ferrous gluconate or glucono delta-lactone.

7. Microemulsion according to claim 1, wherein said liposoluble active ingredient is a vitamin chosen from the group consisting of vitamin D, vitamin K, vitamin A and vitamin E.

8. Microemulsion according to claim 1, further comprising an antioxidant, preferably chosen from the group consisting of caffeic acid, pomegranate plant extracts, rosemary plant extracts, rutin, vitamin E, polyphenols, preferably quercetin, and mixtures thereof.

9. Microemulsion according to claim 1, further comprising an oily phase.

10. Process for the production of a microemulsion according to claim 1, comprising the following steps:
a) mixing, with stirring, at least two surfactants with at least one liposoluble active ingredient until a homogeneous solution is obtained;
b) mixing, with stirring, said homogeneous solution with an aqueous phase until a microemulsion is obtained.

11. Process according to claim 10, further comprising dilution of said microemulsion with an aqueous medium such as water, for example distilled water and/or food-grade water, optionally in admixture with other additives, for example ortho-phosphoric acid and/or citric acid.

12. Process according to claim 10, further comprising an antioxidant, preferably chosen from the group consisting of caffeic acid, pomegranate plant extracts, rosemary plant extracts, rutin, vitamin E, polyphenols, preferably quercetin, and mixtures thereof.

13. Method of making a clear and nutritive microemulsion, comprising: providing an anionic surfactant having an HLB ≧25.

14. Method according to claim 13, wherein said anionic surfactant is chosen from the group consisting of sodium dodecyl sulfate (SDS), alcohol sulfates, alcohol ethoxysulfates, alkylsulfonates and carboxylic acids and their salts, in particular gluconic acid and its derivatives.

15. Method according to claim 13, wherein said anionic surfactant is chosen from the group consisting of gluconic acid and its derivatives such as sodium, potassium, calcium and ferrous gluconate or glucono delta-lactone.

15. A clear and nutritive drink comprising a microemulsion according to claim 1.

16. A clear and nutritive drink according to claim 14, comprising an antioxidant.

17. A clear and nutritive drink according to claim 15, wherein the antioxidant is preferably chosen from the group consisting of caffeic acid, pomegranate plant extracts, rosemary plant extracts, rutin, vitamin E, polyphenols, preferably quercetin, and mixtures thereof.
